(19) United States
(12) Patent Application Publication
(10) Pub. No.: US 2023/0364112 A1
(43) Pub. Date: Nov. 16, 2023

(54) Use of Antcin H and Its Derivatives for Treating Central Nervous System Diseases

(71) Applicant: ALPS BIOTECH CO., LTD., Taipei City (TW)

(72) Inventors: Kuo-Feng Hua, Taipei City (TW); Tz-Chuen Ju, Taipei City (TW); Shui-Tein Chen, Taipei City (TW)

(73) Assignee: ALPS BIOTECH CO., LTD., Taipei City (TW)

(21) Appl. No.: 18/246,797

(22) PCT Filed: Sep. 29, 2021
(86) PCT No.: PCT/CN2021/121859

§ 371 Date: Mar. 27, 2023

Related U.S. Application Data
(60) Provisional application No. 63/084,969, filed on Sep. 29, 2020.

(51) Int. Cl. A61K 31/575; A61P 25/28; A61K 9/00

(52) U.S. Cl. CPC A61K 31/575; A61P 25/28; A61K 9/0053

(57) ABSTRACT

A method of treating central nervous system (CNS) diseases in a subject is provided. The method comprises administering to the subject a therapeutically effective amount of Antcin H and/or its derivatives. The novel use of Antcin H and/or its derivatives are also provided.

This application claims the benefit of U.S. Provisional Application Ser. No. 63/084,969, filed Sep. 29, 2020, the disclosure of which is incorporated by reference herein in its entirety.

### BACKGROUND

### Technical Field

The disclosure relates in general to a method of treating central nervous system in a subject, and more particularly to a method of treating neurodegenerative diseases such as Huntington's disease and/or trinucleotide repeat disorder in a subject.

### Description of the Related Art

Huntington's disease (HD) is an autosomal dominant neurodegenerative disease that manifests clinically as progressive involuntary movement disorders, dementia, and eventual death [1]. It is caused by CAG trinucleotide expansion in exon 1 of the huntingtin (Htt) gene, which is located on the short arm of human chromosome 4 (4p63). When the number of CAG repeats exceeds 36, the translated polyglutamine (polyQ)-containing the Htt protein [mutant Htt (mHtt)] interferes with the normal functions of many cellular proteins and subsequently jeopardizes important cellular machinery [2]. Abnormal accumulation of polyQ-expanded mutant Htt also leads to aggregate formation in the nuclei of neurons, astrocytes, cochlear neurons, and many different types of peripheral cells [3].

Mutant Htt is known to promote protein misfolding and thus inhibit the activity of proteosome activity, dysregulates transcription, impairs synaptic functions, elevates oxidative stress, degenerates axons, and eventually lead to in neurodegeneration and neuronal loss [3]. Extensive release of glutamate from the cortico-striatal terminals and impairment of neuronal survival are believed to account for striatal neurodegeneration which triggers the initial symptoms of HD. Dysfunction of the nigro-striatal pathway also contributes to striatal excitotoxicity. Together, the neuronal degeneration induced by mutant Htt takes place mainly in nonstriatal brain regions (e.g. cortex and substantia nigra) [4, 5], and causes movement disorders, dementia, and eventual death [1, 6].

In addition to neuronal dysregulation, metabolic abnormalities are another important hallmark of HD [7]. Hyperglycemia and abnormal glucose metabolism were observed in several mouse models of HD and in patients with HD [8]. Deficiencies in several other metabolic pathways (e.g., cholesterol biosynthesis and urea cycle metabolism) are also well documented [9, 10]. Deficit of energy metabolism had been proposed as an important pathogenic factor for many neurological disorders. Lately, energy deficit emerged as an important pathogenic factor in HD [7]. Hyperglycemia and reduced insulin had been reported in several transgenic mouse models [8]. Aberrant expressions of proteins associating with glucose metabolism had also been observed in several HD mouse models as well as in HD patients [8]. Although studies of HD have attracted much attention lately, very limited effective treatment for curing HD is not available yet.

Until now, no prior art references report the effect of Antcin H in the HD treatment. The present disclosure using cell experiment (in vitro) proves that Antcin H is a good Inhibitor of the NLRP3 inflammasome, and thus could be used for treating central nervous system diseases such as cerebrovascular diseases (ischemic stroke and hemorrhagic stroke), neurodegenerative diseases (Alzheimer's disease, Huntington's disease, and Parkinson's disease), multiple sclerosis and depression [15, 16, 17].

### REFERENCES

1. Martin J B, Gusella J F: Huntington's disease. Pathogenesis and management. N Engl J Med 1986, 315:1267-1276.
2. Group. THsDCR: A novel gene containing a trinucleotide repeat that is expanded and unstable on Huntington's disease chromosomes. The Huntington's Disease Collaborative Research Group. Cell 1993, 72:971-983.
3. Li H, Li S H, Yu Z X, Shelbourne P, Li X J: Huntingtin aggregate-associated axonal degeneration is an early pathological event in Huntington's disease mice. J Neurosci 2001, 21:8473-8481.
4. Gil J M, Rego A C: Mechanisms of neurodegeneration in Huntington's disease. Eur J

Neurosci 2008, 27:2803-2820.
5. Estrada Sanchez A M, Mejia-Toiber J, Massieu L: Excitotoxic neuronal death and the pathogenesis of Huntington's disease. Arch Med Res 2008, 39:265-276.
6. Vonsattel J P, Myers R H, Stevens T J, Ferrante R J, Bird E D, Richardson E P, Jr.: Neuropathological classification of Huntington's disease. J Neuropathol Exp Neurol 1985, 44:559-577.
7. Pratley R E, Salbe A D, Ravussin E, Caviness J N: Higher sedentary energy expenditure in patients with Huntington's disease. Ann Neurol 2000, 47:64-70.
8. Hurlbert M S, Zhou W, Wasmeier C, Kaddis F G, Hutton J C, Freed C R: Mice transgenic for an expanded CAG repeat in the Huntington's disease gene develop diabetes. Diabetes 1999, 48:649-651.
9. Subhramanyam C S, Wang C, Hu Q, Dheen S T. Microglia-mediated neuroinflammation in neurodegenerative diseases. Semin Cell Dev Biol 2019, 94:112-120.
10. Siew J J, Chen H M, Chen H Y, Chen H L, Chen C M, Soong B W, Wu Y R, Chang C P, Chan Y C, Lin C H, Liu F T, Chern Y. Galectin-3 is required for the microglia-mediated brain inflammation in a model of Huntington's disease. Nat Commun 2019, 10(1):3473.
11. Mangan M S J, Olhava E J, Roush W R, Seidel H M, Glick G D, Latz E. Targeting the NLRP3 inflammasome in inflammatory diseases. Nat Rev Drug Discov 2018, 17(8):588-606.
12. Guo H, Callaway J B, Ting J P. Inflammasomes: mechanism of action, role in disease, and therapeutics. Nat Med 2015, 21(7):677-687.
13. Tschopp J, Schroder K. NLRP3 inflammasome activation: The convergence of multiple signalling pathways on ROS production? Nat Rev Immunol 2010, 10(3):210-215.
14. Ju T C, Chen H M, Lin J T, Chang C P, Chang W C, Kang J J, Sun C P, Tao M H, Tu P H, Chang C, Dickson D W, Chern Y. Nuclear translocation of AMPK-alpha1 potentiates striatal neurodegeneration in Huntington's disease. J Cell Biol 2011, 194(2):209-227.
15. Heneka, M. T., McManus, R. M. & Latz, E. Inflammasome signalling in brain function and neurodegenerative disease. Nat Rev Neurosci 19, 610-621 (2018).
16. Song L, Pei L, Yao S, Wu Y and Shang Y (2017) NLRP3 Inflammasome in Neurological Diseases, from Functions to Therapies. Front. Cell. Neurosci. 11:63.
17. Shao B-Z, Cao Q and Liu C (2018) Targeting NLRP3 Inflammasome in the Treatment of CNS Diseases. Front. Mol. Neurosci. 11:320.

All publications, patents and patent applications referred to herein are incorporated by reference in their entirety to the same extent as if each individual publication, patent or patent application was specifically and individually indicated to be incorporated by reference in its entirety.

### SUMMARY

The disclosure is directed to a new method of treating central nervous system (CNS) diseases in a subject.

According to one embodiment, the method comprises administering to the subject a therapeutically effective amount of Antcin H and/or its derivatives.

In some embodiments, said subject is mammalian or human.

In some embodiments said central nervous system diseases includes cerebrovascular diseases, neurodegenerative diseases, multiple sclerosis and depression.

In some embodiments, said neurodegenerative diseases is Huntington's disease (HD), and said subject has a mutation in the Htt gene and has exhibited at least one symptom of HD.

In some embodiments, said Antcin H and/or its derivatives are naturally derived or synthetic.

In some embodiments, said therapeutically effective amount of Antcin H and/or its derivatives are in a range of from about 0.001 mg/kg/day to about 500 mg/kg/day.

In some embodiments, said Antcin H and/or its derivatives are administering to said subject by at least one route selected from the group consisting of: parenteral, subcutaneous, intramuscular, intravenous, oral, inhalation, rectal, topical, buccal, sub-lingual and transdermal.

In some embodiments, said Antcin H and/or its derivatives are used as inhibitor of the NLRP3 inflammasome.

According to another embodiment, a use of Antcin H and/or its derivatives is provided. The Antcin H and/or its derivatives are used for the manufacture of a medicament for the treatment of CNS diseases.

In some embodiments, said medicament is administered to a subject more than once a day, at least once a day, at least once a week, or at least once a month.

According to an alternative embodiment, a package is provided. The package comprises a pharmaceutical composition comprising one or more unit doses, each such unit dose comprising 0.001-500 mg of Antcin H and/or its derivatives; and instruction for use of the pharmaceutical composition to treat a subject afflicted with CNS diseases.

According to an alternative embodiment, a pharmaceutical composition is provided. The pharmaceutical composition comprises one or more unit doses, each such unit dose comprising 0.001-500 mg of Antcin H and/or its derivatives, for use in treating a subject afflicted with CNS diseases.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

FIG. 1 shows that Antcin H (AH) prevented cell death and reduced oxidative stress in a cell model of HD. FIG. 1A: STHdh^{Q7} and STHdh^{Q109} cells were treated with or without 5 or 10 μM Antcin H for 24 h. The data of indicated cells were normalized to those data of untreated STHdh^{Q7} cells; FIG. 1B: STHdh^{Q7} and STHdh^{Q109} cells were treated with or without 5 or 10 μM Antcin H for 24 h.

FIG. 2 shows that Antcin H (AH) markedly reduced the disease progression of a transgenic mouse model of HD. R6/2 mice or wild-type (WT) mice were treated with AH (10 mg/kg of body weight) or vehicle by oral gavage daily starting from the age of 7 weeks to the age of 12 weeks. FIG. 2A: Body weight; FIG. 2B: rotarod performance; FIG. 2C: survival rate; FIG. 2D: clasping score were measured.

FIG. 3 shows that Antcin H (AH) inhibited neuronal death and mutant huntingtin (mHtt) aggregation in a transgenic mouse model of HD. FIG. 3A: The numbers of neurons (identified by the expression of NeuN; green color) and the level of mutant huntingtin (red color) in the striatum of the mice were analyzed by confocal microscope. Nuclei were stained with Hoechst (blue color). FIG. 3B: The histogram showed the number of striatal neurons; FIG. 3C: The histogram showed the integrated intensity of mHtt; FIG. 3D: Total lysates were collected from the mice brain at age of 12 weeks and NeuN protein expression levels were assessed by Western blotting; FIG. 3E: The level of mHtt aggregates in striatal lysates was analyzed by a filter retardation assay.

FIG. 4 shows Antcin H reduced NLRP3 expression in microglia of R6/2 mice. FIG. 4A: The numbers of activated microglia in the striatum were identified by lba1 staining (green color), and the numbers of NLRP3 positive cells in the striatum were identified by NLRP3 staining (red color). Nuclei were stained with Hoechst (blue color). Double stained microglia is marked with arrows; FIG. 4B: The number of NLRP3 positive cells in striatum region was significantly increased in vehicle-treated R6/2 mice compared to WT mice and also cells were reduced by AH treatment in R6/2 mice.

FIG. 5 shows that Antcin H reduced inflammation in BV2 microglia. FIG. 5A: The IL-1β levels in the supernatants were measured by ELISA; FIG. 5B: The levels of iNOS and NLRP3 in the cell lysates were measured by Western blotting.

FIG. 6 shows that Antcin H inhibited NLRP3 inflammasome in macrophages. FIG. 6A: J774A.1 macrophages were treated with or without 1 μg/ml LPS for 5 h, followed by treated with or without Antcin H for 0.5 h. Cells were then activated by 5 mM ATP for 0.5 h; FIG. 6B: J774A.1 macrophages were treated with or without 1 μg/ml LPS for 5 h, followed by treated with or without 50 μM Antcin H for 0.5 h. Cells were then activated by 5 mM ATP for 0-60 min.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

As will be described in more detail below, the present invention relates to compounds, compositions, methods, packages and/or the like, for treating central nervous system (CNS) diseases in a subject.

The term “subject”, as used herein, refers to any animal who would benefit from treatment and/or diagnosis. Non-limiting examples of a subject includes mammalian animals, such as primates (humans, apes, gibbons, chimpanzees, orangutans, macaques), domestic animals (dogs and cats), farm animals (horses, cattle, goats, sheep, pigs) and experimental animals (mouse, rat, rabbit, guinea pig). In a specific example, the subject is a human.

The term “treatment” or “treating”, as used herein, refers to preventing, reducing, ameliorating, abrogating, delaying disease progression, delaying disease onset, and/or the diminishment of pain. Treatment or treating may further comprise increasing the survival time of a subject suffering from CNS diseases, or increasing the survival time of an individual susceptible to CNS diseases.

Pharmaceutical composition that comprise the combination of the disclosure will typically comprise one or more carriers or excipients and optionally other therapeutic ingredients. The carrier(s) will generally be “acceptable” in the sense of being compatible with the other ingredients of the formulation and physiologically innocuous to the recipient thereof. Such carriers or excipients are known, e.g., fillers, lubricants, binders and various liquid excipients for liquid formulations. Suitable carriers include those disclosed in the references cited herein.

Preferably the combination of this disclosure are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. Alternatively, the combinations may be presented in a form suitable for once-weekly or once-monthly administration. An erodible polymer containing the active ingredient may be envisaged. For preparing solid combinations such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other diluents, e.g. water, to form a solid preformulation combination containing a homogeneous mixture of the compound of the present invention, or a salt, derivative or composition thereof. When referring to these preformulation combinations as homogeneous, it is meant that the ingredient is dispersed evenly throughout the combination so that the combination may be readily subdivided into equally effective unit dosage forms such as tablets, pills and capsules. The tablets or pills of the novel combination can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable compositions include aqueous or oily solutions of the combination of the disclosure. Compositions suitable for parenteral delivery of the active ingredient include aqueous and non-aqueous compositions where the active ingredient is dissolved or suspended in solution. Compositions suitable for parenteral administration include aqueous and non-aqueous sterile injection solutions which may contain anti-oxidants, buffers, bacteriostats or solutes that render the formulation isotonic with the blood of the intended recipient. Other parenteral compositions may comprise aqueous and non-aqueous sterile suspensions which may include suspending agents and thickening agents.

When the CNS diseases is Huntington's disease (HD), delaying disease progression may be indicated by a lack of measurable change in, or an improvement of, one or more indicators of HD, including molecular markers or symptoms of the disease. An improvement in an indicator of HD may include the absence of an undesirable change, or the presence of a desirable change. Treatment or treating may refer to the reduction and/or improvement of any one of the overt symptoms of HD, including, but not limited to, psychiatric, mental cognitive or physical motor impairments. Non-limiting symptoms of HD include, but not limited to, dementia or psychiatric disturbances, ranging from apathy and irritability to bipolar or schizophreniform disorder, physical motor impairment including chorea, hypokinesia, cognitive impairment, motor manifestations including flicking movements of the extremities, a lilting gait, motor impersistence, facial grimacing, ataxia and/or dystonia. It is to be understood that any clinically beneficial effect that arises from the methods, compounds, compositions and kits disclosed herein, is to be considered to be encompassed by the invention.

As used herein, “a subject suffering from CNS diseases” refers to a subject who has CNS diseases. In one example, a subject who has CNS diseases received a diagnosis of CNS diseases from, for example, a health professional, such as a physician. Relevant diagnostic tests are known in the art and include, but are not limited to, genetic testing to determine the presence of a mutation in the huntingtin gene, neurological examination, and brain imaging.

As used herein, “a subject susceptible to CNS diseases” refers to a subject who, based on genetic testing and/or family history, is likely to develop CNS diseases.

Antcin H is a natural triterpene derived from medical fungus Antrodia cinnamomea. The chemical structure of Antcin H is as following:

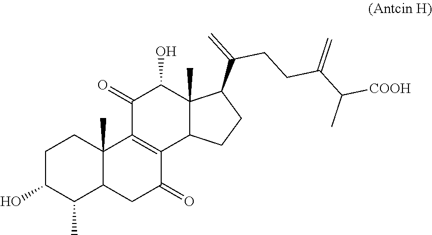 [SMILES: C=C(CCC(=C)[C@H]1CCC2C3=C(C(=O)[C@H](O)[C@@]21C)[C@@]1(C)CC[C@@H](O)[C@@H](C)C1CC3=O)C(C)C(=O)O]

Antcin H has various derivatives, includes but not limited to Antcin A, Antcin B, Antcin C, Antcin D, Antcin E, Antcin F and Antcin K. As The derivatives of Antcin H can be represented by the following formula:

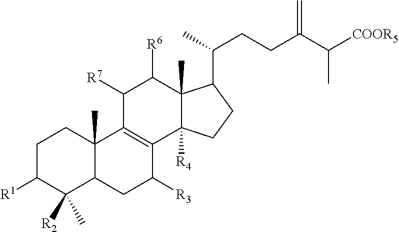

wherein R1 is ═O or OH; R2 is H or OH; R3 is H, ═O, OH or O-acetyl; R4 is H or OH; R5 is H; R6 is H or OH; and R7 is ═O or OH. Both naturally derived or synthetic Antcin H and/or its derivatives could use in the present disclosure.

Examples of routes of administration Antcin H include parenteral (e.g., subcutaneous, intramuscular, intradermal, or intravenous), oral, inhalation (in solid and liquid forms), rectal, topical, buccal (e.g., sub-lingual) and transdermal administration, although the most suitable route in any given case may depend on the nature and severity of the condition being treated and on the nature of the particular form of Antcin H, its derivative, or a pharmaceutically acceptable salt thereof, which is being used.

Compositions of the disclosure suitable for oral administration are prepared as discrete units such as capsules, cachets, gums or tablets each containing a predetermined amount of the low polar fraction of Antcin H and/or its derivatives; as a powder or granules; as solution or a suspension in an aqueous liquid or a non-aqueous liquid; or as an oil-in-water liquid emulsion or a water-in-oil liquid emulsion.

Compositions for rectal administration may be presented as a suppository with a suitable base.

Compositions suitable for intrapulmonary or nasal administration will have a particle size, for example, in the range of 0.01 to 200 microns (including particle sizes in a range between 0.01 and 500 microns in increments of 0.1 microns such as 0.1, 0.2, 0.3, 0.4, 0.5, 1, 2, 5, 30 microns, 35 microns, etc.), which is administered by inhalation through the nasal passage or by inhalation through the mouth so as to reach the various bronchi or alveolar sacs. Formulations suitable for aerosol or dry powder administration may be prepared according to conventional methods and may be delivered with other therapeutic agents.

Compositions suitable for transdermal administration may be presented as transdermal patches. The transdermal patch provides a base line or steady state nicotine level to the patient. The total amount of the low polar fraction of antcin H and/or its derivatives released by the patch during the period of use will vary depending on the user's body size, history of exposure to nicotine, and response to treatment. The size of the patch will vary according to the amount of nicotine to be delivered.

Compositions comprising the combination of the invention are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as described herein.

As used herein, the term “pharmaceutical package” defines an array of one or more unit doses of a pharmaceutical composition, optionally contained wthin common outer packaging. In pharmaceutical package comprising a combination of two or more compounds/agents, the individual compounds/agents may unitary or non-unitary formulations. The unit dose(s) may be contained within a blister pack. The pharmaceutical package may optionally further comprise instructions for use.

The following materials and methods are implemented in the present disclosure:

### Cell Model of HD

Conditionally immortalized wild-type STHdh^{Q7} striatal neuronal progenitor cells expressing endogenous normal htt (referred to as wild-type striatal cells), and homozygous mutant STHdh^{Q109} striatal neuronal progenitor cells from homozygous STHdh^{Q109} knock-in mice expressing mutant htt with 109 glutamines (referred to as mutant striatal cells). STHdh^{Q7} and STHdh^{Q109} cells were generous gifts from Dr. Elena Cattaneo and Marta Valenza (Department of pharmacological sciences and center for stem cell research, University of Milano, Italy). These cells were maintained in an incubation chamber at 33° C. in Dulbecco's modified Eagle's medium supplemented with 10% fetal bovine serum.

### Cell Viability Analysis

STHdh^{Q7} and STHdh^{Q109} cells were treated with 5 or 10 μM Antcin H or vehicle (DMSO) for 24 h. STHdh^{Q7} and STHdh^{Q109} cells viability was quantified by the CCK-8 assay. The survival rate of the indicated cells was normalized to those survival rate of vehicle-treated STHdh^{Q7} cells.

### Intracellular ROS Analysis

STHdh^{Q7} and STHdh^{Q109} cells were treated with 5 or 10 μM Antcin H or vehicle (DMSO) in the presence of 10 μM intracellular ROS indicator fluorescent probe CM-H2DCFDA (Molecular Probes, Inc., Eugene, Oregon, USA) for 1 h. Intracellular ROS (Reactive Oxygen Species) levels were measured by detecting the fluorescence intensity (Excitation/Emission: 488 nm/510 nm) using a fluorescence plate reader (Fluoroskan Ascent; Thermo Electron Corporation, Woburn, MA, USA). J774A.1 macrophages were treated with or without 1 μg/ml LPS for 5 h, followed by treated with or without 50 μM Antcin H for 0.5 h. Cells were then activated by 5 mM ATP for 0-60 min. The levels of intracellular ROS were measured by CM-H2DCFDA staining and detected by a fluorescence plate reader.

### Mice Motor Coordination Ability Assay and Measurement of Body Weight and Lifespan

R6/2 mice or wild-type mice were treated with Antcin H (10 mg/kg of body weight) or vehicle by oral gavage daily starting from the age of 7 weeks to the age of 12 weeks. Mice were tested on a Rotarod apparatus three times a week. Motor coordination was assessed using a rotarod apparatus assay (UGO BASILE, Comerio, Italy) at a constant speed (12 rpm) over a period of 2 min. The body weight and the lifespan were monitored weekly.

The mHtt Aggregation Assay (Filter Retardation Assay)

Brain tissues will be suspended and homogenized in ice-cold RIPA buffer (50 mM Tris-HCl, 0.25% sodium deoxycholate, 1% Triton X-100, 150 mM NaCl, 1 mM phenylmethylsulfonyl fluoride, 1 mM Na3VO4, 0.5 mg/mL aprotinin, 0.1 mM leupeptin, and 4 mM pepstatin), mixed with 2% SDS, and applied to OE66 membrane filters (0.2 mm pore size; Whatman Schleicher and Schuell, Middlesex, UK) through a slot-blot manifold (Bio-Rad, Irvine, CA, USA). The blots will be blocked with 5% skim milk in PBS and incubated overnight with an anti-EM48 antibody (1:500; Millipore Bioscience Research Reagents, Temecula, CA, USA) at 4° C., followed by incubation with the corresponding secondary antibody for 1 h at RT. The immunoreactive bands will be detected by ECL (Pierce, Rockford, IL, USA) and recorded using Kodak XAR-5 film.

### Immunohistochemistry Staining

The method was according to the previous report [14]. In brief, brain sections were incubated overnight with anti-lba-I polyclonal antibody (1:500), anti-NeuN polyclonal antibody (1:500), anti-NLRP3 monoclonal antibody (1:200) or anti-EM48 monoclonal antibody (1:500) in PBS containing 5% normal goat serum at 4° C. and then incubated with the corresponding secondary antibody for 2 h at room temperature. Nuclei were stained with Hoechst 33258. Slides were mounted and analyzed with a laser confocal microscope.

### Activation of Microglia and Macrophages

BV2 microglia were treated with or without 1 μg/ml LPS for 5 h, followed by treated with or without 1 or 5 μM Antcin H for 0.5 h. Cells were then activated by 1 mM ATP for 24 h. The IL-1β levels in the supernatants were measured by ELISA, and the levels of iNOS and NLRP3 in the cell lysates were measured by Western blotting. J774A.1 macrophages were treated with or without 1 μg/ml LPS for 5 h, followed by treated with or without Antcin H for 0.5 h. Cells were then activated by 5 mM ATP for 0.5 h. The IL-1β levels in the supernatants were measured by ELISA.

### Statistical Analysis

The results are expressed as the means±SEM of triplicate samples. Each experiment will be repeated at least three times to confirm the reproducibility of the findings. Multiple groups will be analyzed with one-way analysis of variance (ANOVA), followed by a post-hoc Student-Newman-Keuls test. A p value of <0.05 will be considered significant.

### Example 1: Antcin H Prevented Cell Death and Reduced Oxidative Stress in a Cell Model of HD

To investigate the neuroprotective potential of Antcin H in CNS disease, the present example used striatal cell line as an in vitro model of Huntington's disease. STHdh^{Q109} cells carry the polyQ-expanded mouse gene Htt, which encodes 109 CAG repeats in the Htt gene. Compared with STHdh^{Q109} cells, the control cell line (STHdh^{Q7}) only carry 7 copies of the CAG repeat.

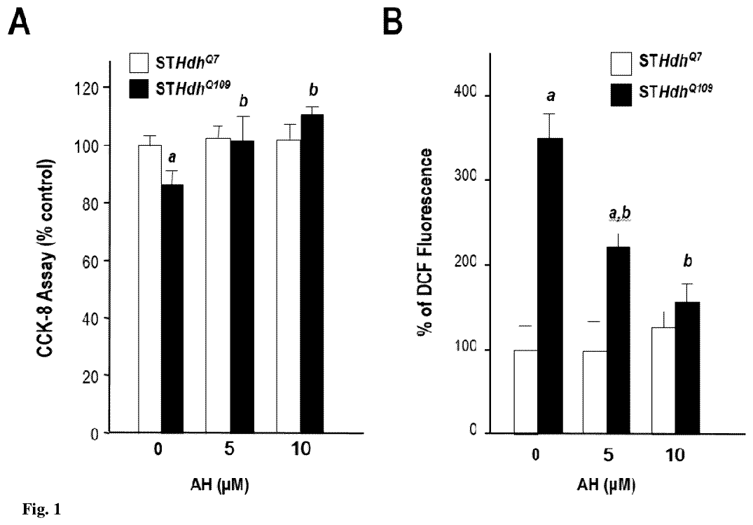

FIG. 1 shows the result of cell survival and intracellular ROS experiment in a cell model of HD. The data are presented as the mean±SEM from three independent experiments. Label “a” in chart means p<0.05 specific comparison between STHdh^{Q7} and STHdh^{Q109} cells. Label “b” in chart means p<0.05 vs. untreated cells.

Experimental shows that treatment with Antcin H significantly prevented STHdh^{Q109} cell death, as 5- and 10-μM Antcin H-treated cells show higher survival ratio then vehicle-treated cells (FIG. 1A). Cell survival was determined using the CCK-8 assay. Additionally, oxidative stress has been demonstrated to be the major cause of cellular injury in HD. The intracellular reactive oxygen species (ROS) levels in STHdh^{Q109} cells were higher than that in STHdh^{Q7} cells (FIG. 1B). The intracellular ROS was measured by dihydrodichlorofluorescein diacetate (H₂DCFDA) staining. Notably, Antcin H significantly reduced the intracellular ROS levels in STHdh^{Q109} cells (FIG. 1B). These results indicate that Antcin H prevented STHdh^{Q109} cell death partially through its anti-oxidative activity.

### Example 2: Antcin H Markedly Reduced the Disease Progression of a Transgenic Mouse Model of HD

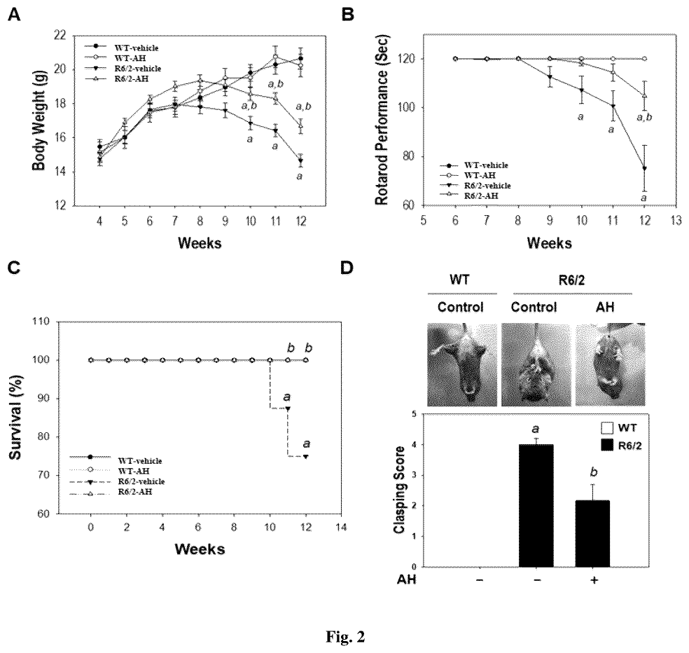

To investigate the therapeutic potential of Antcin H in the pathogenesis of Huntington's disease, R6/2 mice were used as an animal model of HD. R6/2 mice or wild-type mice were treated with Antcin H (10 mg/kg of body weight) or vehicle by oral gavage daily starting from the age of 7 weeks to the age of 12 weeks. FIG. 2 shows that the disease progression of a transgenic mouse model of HD. WT-Vehicle: n=10; WT-AH: n=10; R6/2-vehicle: n=8; R6/2-AH: n=10. The data are presented as the mean±SEM. Label “a” in chart means p<0.05, between WT and R6/2 mice. Label “b” in chart means p<0.05 vs. R6/2-vehicle.

The body weight had monitored weekly and represented as a health condition of mice. It is found that the body weight of vehicle-treated R6/2 mice gradual decreased from 8 to 12 weeks of age, by contrast, the R6/2 mice treated with Antcin H showed a higher weekly weight gain (FIG. 2A). To assess the effects of Antcin H on motor ability such as balance and muscular endurance in R6/2 mice, mice were tested on a Rotarod apparatus three times a week. The rotarod performance of vehicle-treated R6/2 mice gradually decreased since 8 weeks of age. Importantly, an increased latency to fall was observed in Antcin H-treated R6/2 mice (FIG. 2B). In addition, it is found that Antcin H-treated R6/2 mice had 100% survival rate at 12 weeks of age, which is the same with wild-type mice; however, 25% vehicle-treated R6/2 mice died at 12 weeks of age (FIG. 2C). Furthermore, it is found that Antcin H significantly attenuated clasping behavior in R6/2 mice, as Antcin H-treated R6/2 mice had lower clasping score compared with vehicle-treated R6/2 mice at 12 weeks of age (FIG. 2D). These results indicated that Antcin H markedly reduced the disease progression of a transgenic mouse model of HD.

### Example 3: Antcin H Inhibited Neuronal Death and Mutant Huntingtin Aggregation in a Transgenic Mouse Model of HD

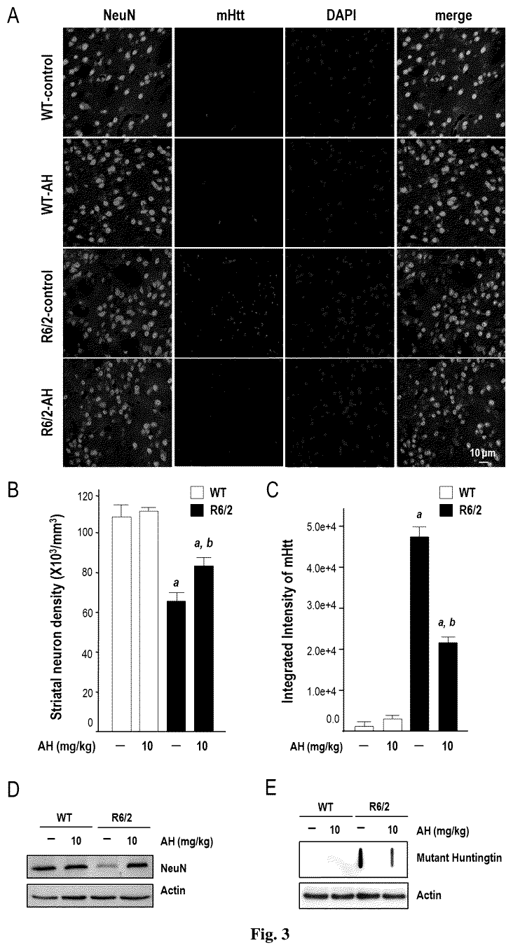

The present example further investigates the effect of Antcin H in the neuronal death of R6/2 mice brains by immunofluorescence staining. FIG. 3 shows that Antcin H (AH) inhibited neuronal death and mutant huntingtin (mHtt) aggregation in a transgenic mouse model of HD. R6/2 mice or WT mice were treated with AH (10 mg/kg of body weight) or vehicle by oral gavage daily starting from the age of 7 weeks to the age of 12 weeks. WT-Vehicle: n=6; WT-AH: n=6; R6/2-vehicle: n=6; R6/2-AH: n=6. The data are presented as the mean±SEM. Label “a” in chart means p<0.05, between WT and R6/2 mice. Label “b” in chart means p<0.05 vs. vehicle-treated R6/2 mice.

Confocal microscope images showed a reduced neuronal cells number in the brain of vehicle-treated R6/2 mice compared to the wild-type mice, indicating the neuronal death increased in R6/2 mice brain (FIGS. 3A and 3B). Notably, the neuronal cells number in the brain of Antcin H-treated R6/2 mice was significant increased compared to the vehicle-treated R6/2 mice (FIGS. 3A and 3B). The neuronal cells increasing effect of Antcin H was confirmed by the increasing expression of NeuN protein in the brain, a neuronal nuclear antigen and neuron differentiation marker (FIG. 3D). Furthermore, mutant huntingtin aggregation in the striatum of vehicle-treated R6/2 mice was significantly increased compared to wild-type mice (FIGS. 3A and 3C). Notably, oral administration of Antcin H reduced the mutant huntingtin aggregation in the striatum of R6/2 mice (FIG. 3A, 3C, 3E). Insoluble aggregates that were retained on the filters were detected using an anti-HTT antibody. These results indicated that Antcin H reduced neuronal death and mutant huntingtin aggregation in a transgenic mouse model of HD.

### Example 4: Antcin H Reduced NLRP3 Expression in Microglia of R6/2 Mice

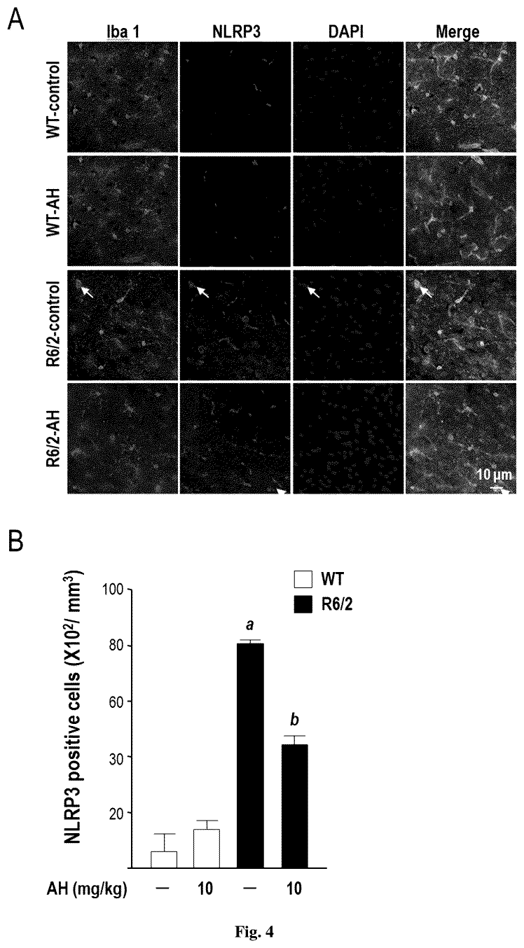

HD commonly associated with inflammation in nervous system and activated microglia. To investigate the anti-inflammatory potential of Antcin H, immunofluorescence staining for striatum was performed to detect the expression levels of lba-1, a protein up-regulated in activated microglia. It is found that the number of lba-1 positive cells was increased in vehicle-treated R6/2 mice compared to WT mice. FIG. 4 show Antcin H reduced NLRP3 expression in microglia of R6/2 mice. R6/2 mice or WT mice were treated with AH (10 mg/kg of body weight) or vehicle by oral gavage daily starting from the age of 7 weeks to the age of 12 weeks. WT-Vehicle: n=6; WT-AH: n=6; R6/2-vehicle: n=6; R6/2-AH: n=6. The data are presented as the mean±SEM. Label “a” in chart means p<0.05, between WT and R6/2 mice. Label “b” in chart means p<0.05 vs. vehicle-treated R6/2 mice.

The number of lba-1 positive cells was decreased in Antcin H-treated R6/2 mice compared to vehicle-treated R6/2 mice (FIG. 4A). In addition, NLRP3 inflammasome plays important role in the inflammatory response and the pathogenesis of neurodegenerative diseases. It is found that the number of NLRP3 positive cells in striatum region was significantly increased in vehicle-treated R6/2 mice compared to WT mice (FIG. 4B). Importantly, Antcin H treatment significantly reduced the number of NLRP3 positive cells in R6/2 mice (FIGS. 4A and 4B).

### Example 5: Antcin H Reduced Inflammation in BV2 Microglia

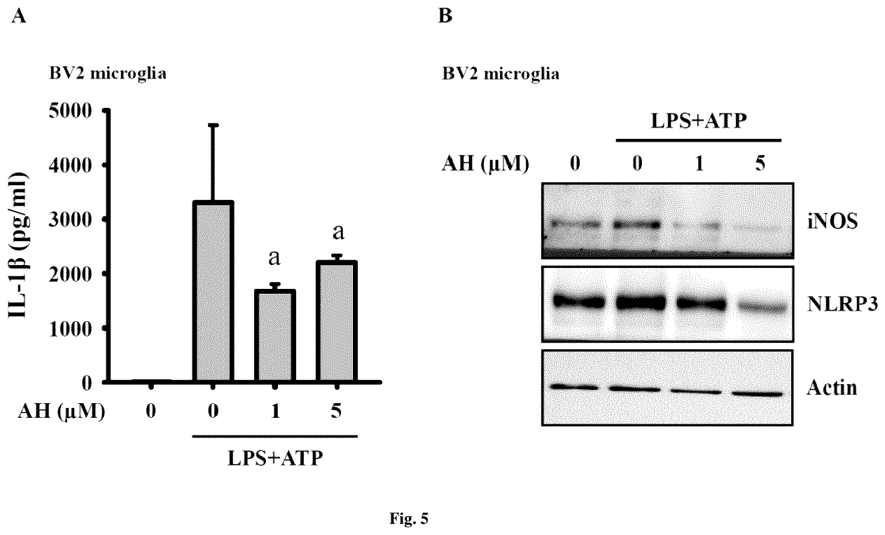

Microglia are macrophage-like immune cells of the central nervous system. It has been demonstrated that neuroinflammation caused by dysregulated activation of microglia promotes the pathogenesis of various neurodegenerative diseases such as Huntington's disease, Alzheimer's disease and Parkinson's disease [9] (Subhramanyam et al., 2019). In addition, overexpressed IL-1β, the end-product of the NLRP3 inflammasome, plays important roles in the development of many diseases including neurodegenerative diseases [10] (Siew et al., 2019). NLRP3 inflammasome composed of NLRP3, ASC and caspase-1 and controls the maturation and release of IL-1β. NLRP3 inflammasome can be activated by medical relevant stimuli such as ATP from damage tissues and amyloid-β of Alzheimer's disease [11] (Guo et al., 2015). FIG. 5A demonstrated that Antcin H reduced ATP-induced IL-1β expression in LPS-primed BV2 microglia, indicating that Antcin H inhibited the NLRP3 inflammasome in BV2 microglia. BV2 microglia were treated with or without 1 μg/ml LPS for 5 h, followed by treated with or without 1 or 5 μM Antcin H for 0.5 h. Cells were then activated by 1 mM ATP for 24 h. The data are presented as the mean±SEM from three independent experiments. Label “a” in chart means p<0.05 vs. LPS+ATP-treated cells.

In addition, Antcin H reduced the expression levels of inducible nitric oxide synthase (iNOS) and NLRP3 in LPS and ATP activated BV2 microglia (FIG. 5B). These results indicated that Antcin H reduced inflammation in BV2 microglia.

Examples 4 and 5 prove that Antcin H is a good Inhibitor of the NLRP3 inflammasome, and thus could be used for treating central nervous system diseases such as cerebrovascular diseases (ischemic stroke and hemorrhagic stroke), neurodegenerative diseases (Alzheimer's disease, Huntington's disease, and Parkinson's disease), multiple sclerosis and depression [15, 16, 17].

### Example 6: Antcin H Reduced IL-1β Expression and ROS Generation in Macrophages

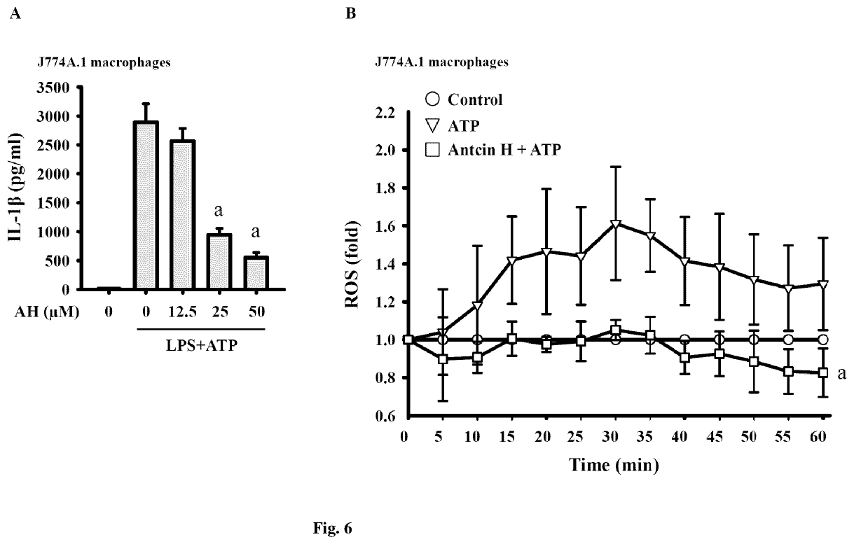

Over-production of IL-113 from NLRP3 inflammasome activated macrophages promotes inflammatory disorders such as neurodegenerative diseases [12] (Mangan et al., 2018). It is found that ATP induces significant IL-18 secretion in LPS-primed macrophages, and this effect was inhibited by Antcin H (FIG. 6A) in a dose-dependent manner. The IL-113 levels in the supernatants were measured by ELISA.

In addition, ROS is one of the crucial elements for NLRP3 inflammasome activation [13] (Tschopp and Schroder, 2010). To determine whether the inhibition of IL-1β secretion by Antcin H occurred via inhibition of ATP-induced ROS production, LPS-primed macrophages were incubated with Antcin H before ATP stimulation. It is found that Antcin H significantly reduced ATP-induced ROS production (FIG. 6B). The levels of intracellular ROS were measured by CM-H2DCFDA staining and detected at an excitation wavelength of 488 nm and an emission wavelength of 510 nm on a microplate absorbance reader. The data are presented as the mean±SEM from three independent experiments. Label “a” in chart means p<0.05 vs. LPS+ATP-treated cells. These results indicated that Antcin H inhibited NLRP3 inflammasome through reducing ROS production in macrophages.

It will be apparent to those skilled in the art that various modifications and variations can be made to the disclosed embodiments. It is intended that the specification and examples be considered as exemplary only, with a true scope of the disclosure being indicated by the following claims and their equivalents.

1. A method of treating central nervous system (CNS) diseases in a subject, comprising administering to the subject a therapeutically effective amount of Antcin H and/or its derivatives.

2. The method of claim 1, wherein said subject is mammalian.

3. The method of claim 2, wherein said subject is human.

4. The method of claim 1, wherein said central nervous system diseases includes cerebrovascular diseases, neurodegenerative diseases, multiple sclerosis and depression

5. The method of claim 4, wherein said neurodegenerative diseases is Huntington's disease (HD), and said subject has a mutation in the Httgene and has exhibited at least one symptom of HD.

6. The method of claim 1, wherein said Antcin H and/or its derivatives are naturally derived or synthetic.

7. The method of claim 1, wherein said therapeutically effective amount of Antcin H and/or its derivatives are in a range of from about 0.001 mg/kg/day to about 500 mg/kg/day.

8. The method of claim 1, wherein said Antcin H and/or its derivatives are administering to said subject by at least one route selected from the group consisting of: parenteral, subcutaneous, intramuscular, intravenous, oral, inhalation, rectal, topical, buccal, sub-lingual and transdermal.

9. The method of claim 1, wherein said Antcin H and/or its derivatives are used as inhibitor of the NLRP3 inflammasome.

10. (canceled)

11. (canceled)

12. (canceled)

13. (canceled)

14. (canceled)

15. A package comprising:
a) a pharmaceutical composition comprising one or more unit doses, each such unit dose comprising 0.001-500 mg of Antcin H and/or its derivatives; and
b) instruction for use of the pharmaceutical composition to treat a subject afflicted with CNS diseases.

16. A pharmaceutical composition comprising one or more unit doses, each such unit dose comprising 0.001-500 mg of Antcin H and/or its derivatives, for use in treating a subject afflicted with CNS diseases.
